(19) United States
(12) Patent Application Publication
(10) Pub. No.: US 2025/0327030 A1
(43) Pub. Date: Oct. 23, 2025

(54) A METHOD FOR PRODUCING iPS CELL-DERIVED NATURAL KILLER CELLS

(71) Applicant: Kyoto University, Kyoto (JP)

(72) Inventors: Shin Kaneko, Kyoto (JP); Shoichi Iriguchi, Kyoto (JP); Tatsuki Ueda, Kyoto (JP)

(21) Appl. No.: 18/877,439

(22) PCT Filed: Jun. 22, 2023
(86) PCT No.: PCT/JP2023/023076

§ 371 Date: Dec. 20, 2024

(30) Foreign Application Priority Data
Jun. 27, 2022 (JP) 2022-102871

(51) Int. Cl. C12N 5/0783; A61K 40/15; A61K 40/31; A61K 40/42; C07K 16/30

(52) U.S. Cl. CPC C12N 5/0646; A61K 40/15; A61K 40/31; A61K 40/4261; C07K 16/303; C12N 2500/25; C12N 2500/38; C12N 2501/115; C12N 2501/125; C12N 2501/145; C12N 2501/165; C12N 2501/21; C12N 2501/2307; C12N 2501/26; C12N 2501/727; C12N 2506/45; C12N 2510/00

(57) ABSTRACT

The present invention provides a method for producing a natural killer cell from an iPS (Induced pluripotent stem) cell, comprising steps of: (i) contacting an iPS cell with a composition comprising a GSK-3 inhibitor and a ROCK inhibitor to obtain an embryoid body, (ii) contacting the embryoid body with a composition comprising a TGFβ receptor inhibitor to obtain a hematopoietic progenitor cell, (iii) culturing the hematopoietic progenitor cell to obtain a lymphocyte progenitor cell, and (iv) differentiating and expanding the lymphocyte progenitor cells to a natural killer cell.

### TECHNICAL FIELD

The present invention relates to a method for producing d a natural killer cell from an iPS (Induced pluripotent stem) cell, a natural killer cell or a population, a pharmaceutical composition comprising the natural killer cell or the population and a method for treating cancer, comprising administrating the pharmaceutical composition comprising the natural killer cell or the population.

### BACKGROUND ART

Natural killer (NK) cells are cytotoxic lymphocytes that constitute a major component of the innate immune system. Natural killer (NK) cells are a subset of innate lymphoid cells (ILCs) that have direct cytotoxic effects on pathogenic cells by inducing apoptosis.

NK cells are activated in response to interferons or macrophage-derived cytokines. The cytotoxic activity of NK cells is largely regulated by two types of surface receptors, which may be considered “activating receptors” or “inhibitory receptors” although some receptors, e.g., CD94 and 2B4 (CD244), can work either way depending on ligand interactions.

Among other activities, NK cells play a role in the host rejection of tumors and have been shown capable of killing virus-infected cells. Natural killer cells can become activated by cells lacking, or displaying reduced levels of, major histocompatibility complex (MHC) proteins. Cancer cells with altered or reduced level of self-class I MHC expression result in induction of NK cell sensitivity. Activated and expanded NK cells, and in some cases LAK cells, from peripheral blood have been used in both ex vivo therapy and in vivo treatment of patients having advanced cancer, with some success against bone marrow related diseases, such as leukemia; breast cancer; and certain types of lymphoma.

Recently, anti-GPC3 CAR-expressing NK/ILC cells, which show an effective cell therapy against disseminated ovarian tumors have been reported (Cancer Sci. 2020 May; 111(5):1478-1490. doi: 10.1111/cas. 14374. Epub 2020 Mar. 31.).

### SUMMARY OF INVENTION

### Technical Problem

In spite of the advantageous properties of NK cells in killing tumor cells and virus-infected cells, they remain difficult to apply in immunotherapy, primarily due to the difficulty in maintaining their tumor-targeting and tumoricidal capabilities during culture and expansion. Thus, there is a need in the art to develop an efficient method to produce and expand natural killer cells that retain tumoricidal functions.

### Solution to Problem

The present invention provides a method for producing a natural killer cell from an iPS (Induced pluripotent stem) cell, a natural killer cell or a population, a pharmaceutical composition comprising the natural killer cell or the population and a method for treating cancer, comprising administrating the pharmaceutical composition comprising the natural killer cell or the population.

Specifically, the following inventions are provided.
1. A method for producing a natural killer cell from an iPS (Induced pluripotent stem) cell, comprising steps of:
(i) contacting an iPS cell with a composition comprising a GSK-3 inhibitor and a ROCK inhibitor to obtain an embryoid body,
(ii) contacting the embryoid body with a composition comprising a TGFβ receptor inhibitor to obtain a hematopoietic progenitor cell,
(iii) culturing the hematopoietic progenitor cell to obtain a lymphocyte progenitor cell, and
(iv) differentiating and expanding the lymphocyte progenitor cell to a natural killer cell.
2. The method according to 1, wherein the iPS cell expresses a tumor antigen specific chimeric antigen receptor (CAR).
3. The method according to 2, wherein the CAR expression is maintained or selected during differentiation process using a tracer gene and the CAR is stably expressed at the natural killer cell stage.
4. The method according to 2, wherein the tumor antigen is selected from a group consisting of GPC3, BCMA, PSMA, MUC1, HER2, Mesothelin, Lewis-Y, AXL, EGFR, Claudin18.2, B7-H3, NKG2D, GD2, EpCAM, ROBO-1, CD19, CD20, CD22, CD30, CD33, CD38, CD123, CD276, and CD269.
5. The method according to 2, wherein the iPS cell is undifferentiated from iPS cell colonies with CAR.
6. The method according to 2, wherein the CAR is transduced into an iPS cell using viral vectors, non-viral vectors, artificial chromosomes, or gene editing.
7. The method according to 6, wherein the viral vectors are Lentiviral vectors, retroviral vectors, adenoviral vectors or AAV vectors, and the non-viral vectors are piggyBac vectors.
8. The method according to 6, wherein the gene editing comprises using CRISPAR/CAS9, Talen, homologous recombination, or other gene editing tools.
9. The method according to 1, wherein the GSK-3 inhibitor is CHIR99021 and the ROCK inhibitor is Y-27632.
10. The method according to 1, wherein the TGFβ receptor inhibitor is SB431542.
11. The method according to claim 1, wherein the composition in step 2 further comprising VEGF, hbFGF and SCF.
12. The method according to 1, wherein the hematopoietic progenitor cell is cultured with a composition comprising 2-mercaptoethanol, insulin-transferrin-selenium, ascorbic acid-2-phosphate, SCF, TPO, IL-7, hFlt3L, SDF1α, and p38 inhibitor.
13. The method according to 1, wherein the p38 inhibitor is SB203580.
14. The method according to 1, wherein the lymphocyte progenitor cell is CD7+CD45+ cells.
15. The method according to 1, wherein the lymphocyte progenitor cell is expanded on a feeder cell comprising a human PBMC.
16. The method according to 15, wherein the human PBMC is autologous or allogeneic.
17. A natural killer cell or a population thereof, produced by the method according to any one of 1 to 16.
18. A natural killer cell population, comprising cells that are CD7 CD45 cells.
19. The natural killer cell population according to 18, wherein a percentage of CD7+CD45+ cells in the natural killer cell is more than 60% by cell number.
20. The natural killer cell population according to 18, wherein the cells are CD3⁻, CD4⁻, CD5⁻, CD8⁻, CD117⁺, CD337⁺, CD159a⁺, CD161⁺, CD336⁺, CD226⁺, and CD314⁺.
21. The natural killer cell population according to 18, wherein a contamination of undifferentiated iPSC is less than 0.01% by cell number in the natural killer cell.
22. A pharmaceutical composition comprising the natural killer cell or the population thereof according to any one of 17 to 21.
23. The pharmaceutical composition according to 22, comprising a cryoprotective agent.
24. The pharmaceutical composition according to 22, comprising glucose, saline, dextran D, albuminar and dimethyl sulfoxide.
25. A method for treating cancer, comprising administrating the pharmaceutical composition according to any one of 22 to 24.
26. The method according to 25, wherein the cancers are liver cancers, ovarian cancer, gastric cancers, lung cancers, prostate cancers, breast cancers, glioblastoma, colorectal cancers, esophageal cancers, head and neck cancers, cervical cancers, renal cancers, pediatric solid tumors, osteosarcoma, germ cell tumors, neuroblastoma, hematological malignancies, or multiple myeloma.

### BRIEF DESCRIPTION OF DRAWINGS

FIG. 1 shows Cytotoxic activities of iCAR-ILC/N101. Cytotoxic activities of iCAR-ILC/N101 were examined using the 51Cr release assay. Ovarian cancer KOC7c cells endogenously expressing GPC3 were used as target cells. Significant effector/target ratio-dependent cytotoxic activity against KOC7c was observed.

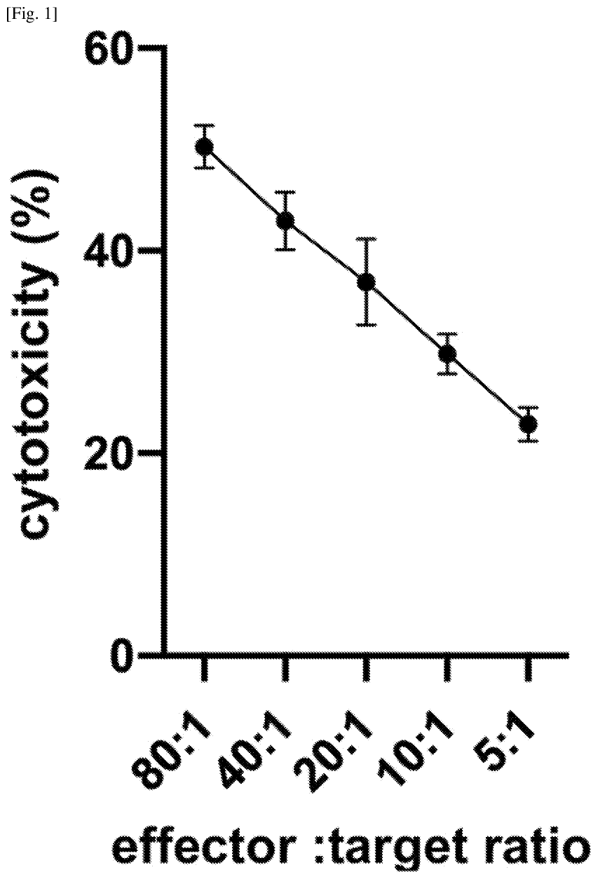

FIG. 2 shows cytokine dependent growth of ICAR-ILC/N101. iCAR-ILC/N101 cells were stimulated with PHA-P (1 μg/ml) and irradiated PBMC (iCAR-ILC/N101 cells:PBMC=1:10) for three days. The cells were cultured with or without IL7 (10 ng/ml) and IL15 (5 ng/ml). The cell numbers were examined every three days. iCAR-ILC/N101 cells demonstrated significant cytokine dependent growth as shown in the FIG. 2. No cytokine independent growth was seen.

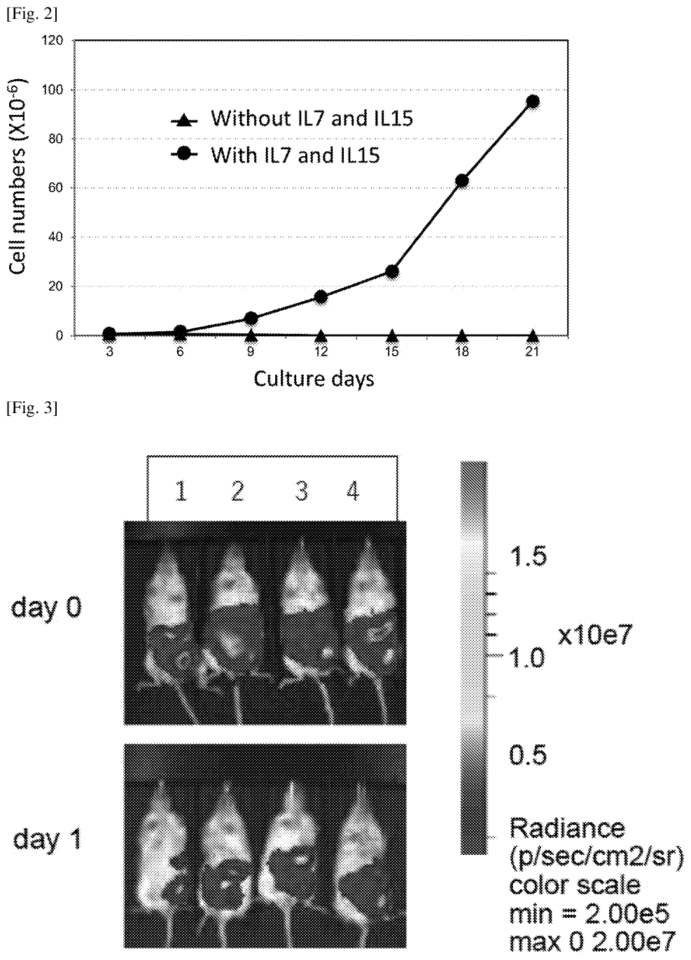

FIG. 3 and FIG. 4 shows pharmacokinetics of iCAR-ILC/N101. Luciferase gene-transduced ICAR-ILC/N101 cells were inoculated in peritoneal cavity of NOG mice and their chemiluminescence was monitored at different rime points using an in vivo bioluminescence imaging instrument. The chemiluminescence of ICAR-ILC/N101 was detected up to 7 days after inoculation. No significant chemiluminescence was seen on days 14, 21, and 28.

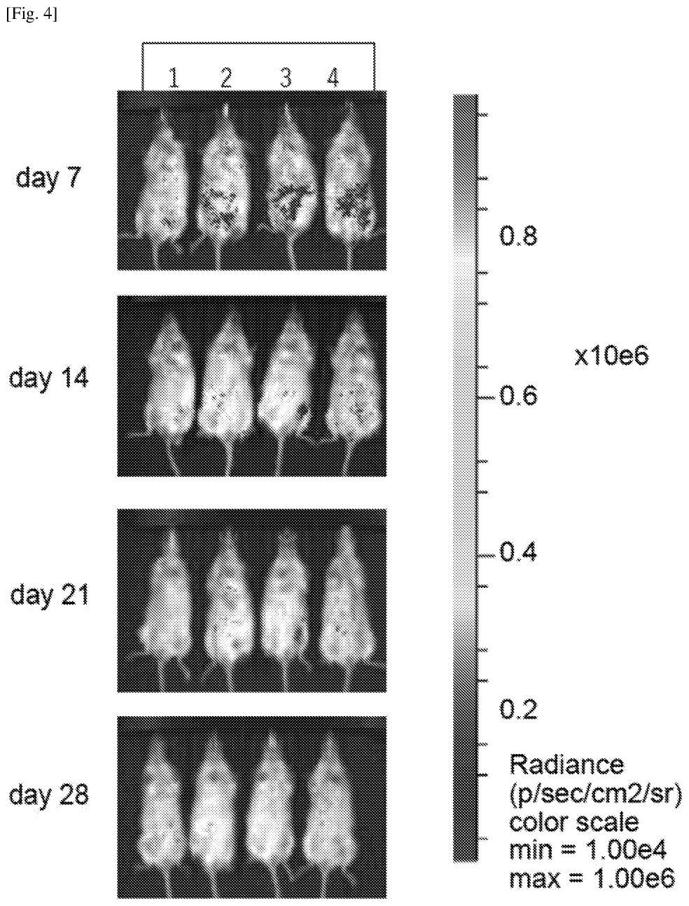

FIG. 5 shows contamination of iPS cells in iCAR-ILC/N101. iPSC (iPS cells) contamination was examined using qPCR detecting LIN28A RNA. Cell lysates were prepared from 1×10⁶ iCAR-ILC-N101, 1×10⁶ human MSC (hMSC), and mixture of 1×10⁶ iPS cells and 1×10⁶ human MSC. The lysate of the iPS cells and human MSC mixture was serially 10-fold diluted in the human MSC lysate. The following standard lysate solutions were made:

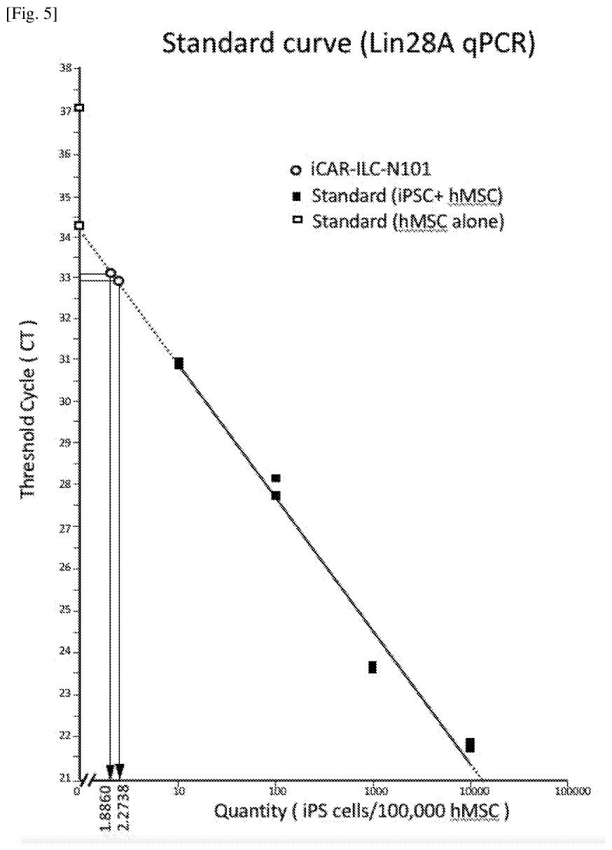

$$iPSC : hMSC = \begin{matrix}\begin{matrix}\begin{matrix}1 : 10 & 1 : 100\end{matrix} & 1 : 1000\end{matrix} & 1 : 10000\end{matrix}$$

qPCR was performed using the total RNA prepared from the lysates of iCAR-ILC-N101, human MSC, and the mixture of iPS cells and human MSC. The standard line was drawn based upon the CT values of the mixture of iPS cells and human MSC. The quantity was calculated for the iCAR-ILC-N101 samples. The values were out of the range (0.01%˜10%). Therefore, the contamination rate was concluded as <0.01.

### DESCRIPTION OF EMBODIMENTS

### Best Mode for Carrying Out the Invention

In one embodiment, the present invention discloses a method for producing a natural killer cell from an iPS (Induced pluripotent stem) cell comprising the steps of:
(step i) contacting iPS cells with a composition comprising a GSK-3 inhibitor and a ROCK inhibitor to obtain an embryoid body,
(step ii) contacting the embryoid body with a composition comprising a TGFβ receptor inhibitor to obtain a hematopoietic progenitor cell,
(step iii) culturing the hematopoietic progenitor cell to obtain a lymphocyte progenitor cell, and
(step iv) differentiating and expanding the lymphocyte progenitor cells to a natural killer cell.

The iPS cells are tumor antigen specific chimeric antigen receptor (CAR)-transduced iPS cells. CAR expression is maintained/selected during differentiation process using a tracer gene and CAR is stably expressed at the natural killer cell stage.

Tumor antigen is GPC3, BCMA, PSMA, MUC1, HER2, Mesothelin, Lewis-Y, AXL, EGFR, Claudin18.2, B7-H3, NKG2D, GD2, EpCAM, ROBO-1, CD19, CD20, CD22, CD30, CD33, CD38, CD123, CD276, or CD269.

The iPS cells are undifferentiated CAR-transduced iPSC colonies. The CAR is transduced into iPS cells using viral vectors, non-viral vectors, artificial chromosomes, or gene editing.

The examples of the viral vectors are Lentiviral vectors, retroviral vectors, adenoviral vectors or AAV vectors, the non-viral vectors are piggyBac vectors.

The examples of the gene editing are CRISPAR/CAS9, Talen, homologous recombination, or other gene editing tools.

A GSK-3 inhibitor can maintain or increase cell's capacity to differentiate (potency) to a greater extent than cells cultured in the absence of a GSK-3 inhibitor. Examples of GSK-3 inhibitor include SB216763, AT7519, CHIR-98014, TWS119, SB415286, NP031112, BIO, preferably CHIR99021.

A ROCK inhibitor can increase proliferation of cells to a greater extent than cells cultured in the absence of a ROCK inhibitor. Examples of ROCK inhibitors include ZINC00881524, Thiazovivin, Fasudil, GSK429286A, RKI-1447, NSC 33669, GSK269962, AR-13324, TC-S 7001, Y-33075, KD025, HA-1100, H-1152 dihydrochloride, AT13148, preferably Y-27632.

Examples of TGFβ receptor inhibitors include LY2157299, LY2109761, SB525334, SB505124, GW788388, LY364947, preferably SB431542.

In one embodiment, the present invention discloses a method for producing a natural killer cell from an iPS (Induced pluripotent stem) cell, further comprising VEGF, hbFGF and SCF in step 2.

In one embodiment, the present invention discloses a method for producing a natural killer cell from an iPS (Induced pluripotent stem) cell, wherein the hematopoietic progenitor cell is cultured with a composition comprising 2-mercaptoethanol, insulin-transferrin-selenium, ascorbic acid-2-phosphate, SCF, TPO, IL-7, hFlt3L, SDF1α, and p38 inhibitor, preferably the p38 inhibitor is SB203580. The lymphocyte progenitor cells are CD7⁺CD45⁺ cells.

In one embodiment, the present invention discloses a method for producing a natural killer cell from an iPS (Induced pluripotent stem) cell, wherein the lymphocyte progenitor cell is expanded on a feeder cell comprising human PBMC. Preferably the human PBMC is autologous or allogeneic.

In one embodiment, the present invention discloses a natural killer cell or a population thereof, produced by the present method.

In one embodiment, the present invention discloses a natural killer cell population, comprising cells that are CD7⁺CD45⁺ cells, preferably, a percentage of CD7⁺CD45⁺ cells in the natural killer cell is more than 60% by cell number.

In one embodiment, the present invention discloses a natural killer cell population, containing cells that are CD3⁻, CD4⁻, CD5⁻, CD8⁻, CD117⁺, CD337⁺, CD159a⁺, CD161⁺, CD336⁺, CD226⁺, and CD314⁺.

In one embodiment, the present invention discloses a natural killer cell population, preferably, a contamination of undifferentiated iPSC is less than 0.01% by cell number in the natural killer cell.

In one embodiment, the present invention discloses a pharmaceutical composition comprising the natural killer cell or the population thereof. The pharmaceutical composition further comprises a cryoprotective agent such as glucose, saline, dextran D, albuminar and dimethyl sulfoxide.

In one embodiment, the present invention discloses a method for treating cancer, comprising administrating the pharmaceutical composition comprising the natural killer cell or the population thereof.

Cancers are liver cancers, ovarian cancer, gastric cancers, lung cancers, prostate cancers, breast cancers, glioblastoma, colorectal cancers, esophageal cancers, head and neck cancers, cervical cancers, renal cancers, pediatric solid tumors, osteosarcoma, germ cell tumors, neuroblastoma, hematological malignancies, or multiple myeloma.

### DETAILED DESCRIPTION OF THE INVENTION

The examples of the invention that are described below are simply provided as examples, and shall not limit the technical scope of the present invention. The technical scope of the present invention is only limited by the descriptions in the scope of claims. The present invention may be modified, for example, elements may be added to the present invention, and the elements of the invention may also be deleted or even substituted without departing from the gist of the present invention.

Conditions that are not specified in the examples will be the common conditions in the art or the recommended conditions of the raw materials by the product manufacturer. The reagents which are not indicated the origin will be the commercially available conventional reagents.

Natural killer cell (NK)/Innate Lymphoid cell (ILC) production method (Methods for manufacturing iPSC-derived NK/ILCs)

Tumor antigen specific chimeric antigen receptor (CAR)-transduced iPS cells were differentiated into a hematopoietic precursor through the feeder-free embryoid body (EB) formation method as described below.

### Step 1

Undifferentiated CAR-transduced iPSC colonies were treated with TrypLE select (Gibco) for 4 minutes (up to 10 minutes, this process depends on how quickly cells get dispersed), transferred to low-attachment plates, and incubated overnight in Medium A (StemFit AK03N supplemented with 10 μmol/L ROCK inhibitor (Y-27632) and 10 μmol/L GSK3b inhibitor (CHIR99021)) to allow for the formation of EBs.

TABLE 1
Medium A

| No. | Content | Final concentration |
|---|---|---|
| 1 | StemFit AK03N | — |
| 2 | Y-27632 | 10 μmol/L |
| 3 | CHIR99021 | 10 μmol/L |

### (Step 2)

The EBs were collected, centrifuged, and resuspended in Medium B [StemPro-34 supplemented with 2 mmol/L L-glutamine (1% GlutaMAX), 400 μmol/L monothioglycerol, 50 μg/mL ascorbic acid-2-phosphate, 1% insulin-transferrin-selenium supplement, 50 ng/ml hBMP-4, 50 ng/mL hbFGF, and 50 ng/mL VEGF] followed by incubation at 37° C. in 5% CO₂ atmosphere.

TABLE 2
Medium B

| No. | Content | Final concentration | |
|---|---|---|---|
| 1 | StemPro-34* | — | |
| 2 | GlutaMAX (100x) | 1x (1%) | |
| 3 | monothioglycerol | 400 | μmol/L |
| 4 | ascorbic acid-2-phosphate | 50 | μg/mL |
| 5 | Insulin-transferrin-selenium (100x) | 1x (1%) | |
| 6 | hBMP-4 | 50 | ng/mL |
| 7 | hbFGF | 50 | ng/mL |
| 8 | VEGF | 50 | ng/mL |

*StemPro-34 does not contain TGF-beta, IL-3, GSK3 inhibitors, or ROCK inhibitors.

### (Step 3)

On day 2, 6 μmol/L TGFβ receptor inhibitor (SB431542) was added to the culture.

### (Step 4)

On day 4, the EBs were collected, centrifuged, and resuspended in Medium C (StemPro-34 supplemented with 2 mmol/L L-glutamine, 400 μmol/L monothioglycerol, 50 μg/mL μg/mL ascorbic acid-2-phosphate, 1% insulin-transferrin-selenium supplement, 50 ng/mL hbFGF, 50 ng/mL VEGF, and 50 ng/mL SCF) followed by incubation at 37° C. in 5% CO₂ atmosphere.

TABLE 3
Medium C

| No. | Content | Final concentration | |
|---|---|---|---|
| 1 | StemPro-34 | — | |
| 2 | GlutaMAX (100x) | 1x (1%) | |
| 3 | monothioglycerol | 400 | μmol/L |
| 4 | ascorbic acid-2-phosphate | 50 | μg/mL |
| 5 | Insulin-transferrin-selenium (100x) | 1x (1%) | |
| 6 | hbFGF | 50 | ng/mL |
| 7 | VEGF | 50 | ng/mL |
| 8 | SCF | 50 | ng/mL |

### (Step 5)

On days 6, 8, 11, and 13, cells in the culture were collected, centrifuged, and resuspended in Medium D (StemPro-34 supplemented with 2 mmol/L L-glutamine, 400 μmol/L monothioglycerol, 50 μg/mL ascorbic acid-2-phosphate, 1% insulin-transferrin-selenium supplement, 50 ng/mL hbFGF, 50 ng/ml, 50 ng/mL VEGF, 50 ng/mL SCF, 100 ng/ml TPO, and 50 ng/mL hFlt3L,) followed by incubation at 37° C. in 5% CO₂ atmosphere.

TABLE 4
Medium D

| No. | Content | Final concentration | |
|---|---|---|---|
| 1 | StemPro-34 | — | |
| 2 | GlutaMAX (100x) | 1x (1%) | |
| 3 | monothioglycerol | 400 | μmol/L |
| 4 | ascorbic acid-2-phosphate | 50 | μg/mL |
| 5 | Insulin-transferrin-selenium (100x) | 1x (1%) | |
| 6 | hbFGF | 50 | ng/mL |
| 7 | VEGF | 50 | ng/mL |
| 8 | SCF | 50 | ng/mL |
| 9 | TPO | 100 | ng/mL |
| 10 | hFlt3L | 50 | ng/mL |

### (Step 6)

On day 14, single cell suspension was prepared using a cell strainer, and transferred onto FcDLL4-coated plates. The cells were cultured in Medium E (α-MEM supplemented with 15% FBS, 55 μM 2-mercaptoethanol, 1% insulin-transferrin-selenium, 50 μg/mL ascorbic acid-2-phosphate, 50 ng/mL SCF, 100 ng/ml TPO, 10 ng/ml IL-7, 50 ng/ml hFlt3L, 240 ng/ml SDF1α, and 15 μM p38 inhibitor (SB203580)).

TABLE 5
Medium E

| No. | Content | Final concentration | |
|---|---|---|---|
| 1 | a-MEM | — | |
| 2 | FBS | 15% | |
| 3 | 2-mercaptoethanol | 55 | μM |
| 4 | Insulin-transferrin-selenium (100x) | 1x (1%) | |
| 5 | ascorbic acid-2-phosphate | 50 | μg/mL |
| 6 | SCF | 50 | ng/mL |
| 7 | TPO | 100 | ng/mL |
| 8 | IL-7 | 10 | ng/mL |
| 9 | hFlt3L | 50 | ng/mL |
| 10 | SDF1α | 240 | ng/mL |
| 11 | p38i (SB203580) | 15 | μM |

On days 15, 18, 22, 25, 29, and 32, cells in the culture were collected, centrifuged, resuspended in fresh Medium E, and transferred back to the same culture vessels. On days 21 and 28, the cells were collected, centrifuged, resuspended in fresh Medium E, and transferred onto new FcDLL4-coated plates.

### (Step 7)

On day 35, after 21 days of culture, the hematopoietic cells were differentiated into CD7, CD45-positive lymphocyte progenitor cells.

### (Step 8)

Floating cells were recovered and passed through a cell strainer, and the plates where cells remained were washed with PBS. The floating cells and the cell-containing PBS solution were mixed, centrifuged, and resuspended in STEM-CELLBANKER (Registered Trademark). The cells were frozen-stored.

### (Step 9)

The cells and the frozen-stored irradiated human peripheral mononuclear cells (PBMC) were thawed, centrifuged, and resuspended in Medium F [α-MEM supplemented with 15% FBS, 1× (1%) insulin-transferrin-selenium, 50 μg/mL ascorbic acid-2-phosphate, 10 ng/ml IL-7, 5 ng/ml IL-15, and 2 μg/mL Phytohemagglutinin (PHA)]. The cells and the PBMC were mixed in the ratio 1:14 and cultured for 10˜16 days. IL-15 and IL-7 are used as a key raw material for NK cell activation and amplification.

TABLE 6
Medium F

| No. | Content | Final concentration | |
|---|---|---|---|
| 1 | α-MEM | — | |
| 2 | FBS | 15% | |
| 3 | Insulin-transferrin-selenium (100x) | 1x (1%) | |
| 4 | ascorbic acid-2-phosphate | 50 | μg/mL |
| 5 | IL-7 | 10 | ng/mL |
| 6 | IL-15 | 5 | ng/mL |
| 7 | PHA | 2 | μg/mL |

Every 2˜3 days during 10˜16-day culture, the culture medium was replaced with fresh medium G [α-MEM supplemented with 15% FCBS, 1× (1%) insulin-transferrin-selenium, 50 μg/mL ascorbic acid-2-phosphate, 10 ng/mL IL-7, and 105 ng/ml IL-15]. When cells are were growing well, the culture was split into two and replenished with fresh medium G. When sufficient growth was not observed, a half of the culture was collected, centrifuged, resuspended in fresh Medium G and back to the original plates.

TABLE 7
Medium G

| No. | Content | Final concentration | |
|---|---|---|---|
| 1 | α-MIEM | — | |
| 2 | FBS | 15% | |
| 3 | Insulin-transferrin-selenium (100x) | 1x (1%) | |
| 4 | ascorbic acid-2-phosphate | 50 | μg/mL |
| 5 | IL-7 | 10 | ng/mL |
| 6 | IL-15 | 5 | ng/mL |

After the 10˜16-day culture, the cells were harvested, washed three times with PBS by centrifugations, resuspended in Cryoprotective agent A as the final product (iCAR-ILC/N101), and frozen-stored until just before using.

TABLE 8
Cryoprotective agent A

| No. | Content | Volume | |
|---|---|---|---|
| 1 | Physio140 Injection | 62.5 | mL |
| 2 | OTSUKA GLUCOSE INJECTION | 31.25 | mL |
| 3 | OTSUKA NORMAL SALINE | 31.25 | mL |
| 4 | LOW MOLECULAR DEXTRAN D | 20 | mL |
| | INJECTION | | |
| 5 | ALBUMINAR 5% I.V. INJECTION | ALBUMINAR I.V.I | |
| 6 | Dimethyl sulfoxide (DMSO) | 15 | mL |

Stability of Frozen-Stored iCAR-ILC/N101

Table 9 shows long term stability of frozen-stored iCAR-ILC/N101.

TABLE 9

| Test items | Day 0 | | | Day 43 | | | Day 57 | | | Day 71 | | | Day 137 | | |
|---|---|---|---|---|---|---|---|---|---|---|---|---|---|---|---|
| Tube Number | 1 | 28 | 71 | 18 | 36 | 67 | 46 | 32 | 39 | 66 | 40 | 48 | 55 | 49 | 65 |
| Viability (%) | 84 | 84 | 85 | 81 | 75 | 77 | 84 | 84 | 79 | 80 | 82 | 84 | 77 | 81 | 80 |
| Cell conc. | 1.84 | 2.00 | 1.89 | 1.82 | 1.72 | 1.79 | 1.87 | 1.90 | 1.81 | 1.86 | 1.93 | 1.93 | 1.62 | 1.72 | 1.86 |
| (×10⁻⁷/ml) | | | | | | | | | | | | | | | |
| CAR | 99.6 | | | 99.6 | | | 99.3 | | | 98.8 | | | | | 98.9 |
| positivity (%) | | | | | | | | | | | | | | | |
| Product purity | 99.5 | | | 99.4 | | | 98.1 | | | 99.1 | | | | | 98.9 |
| (%) | | | | | | | | | | | | | | | |
| IFN-γ production | | 514.7 | | | 155.8 | | | 325.19 | | | 1142 | | 528.03 | | |
| (MFI) | | 445.0 | | | | | | | | | | | | | |
| Endotoxin | Negative | | | | | | | | | Negative | | | Negative | | |
| Mycoplasma | | | | | | | | | | | | | | | |
| Bacteria | | | | | | | | | | | | | | | |

| | | | |
|---|---|---|---|
| Appearance | Nothing in particular | Nothing in particular | Nothing in particular |

iCAR-ILC/N101 cells are aliquoted as 2×107 cells/tube and kept frozen in the gas phase of a liquid nitrogen tank for 137 days. On days 0, 43, 57, 71, and 137, viability, cell concentration, CAR positivity, product (NK) purity, IFN-γ production, endotoxin/mycoplasma/bacterial pathogen detection, and appearance were examined. All test results passed the quality standard in the table 9.

Table 10 shows transport stability of frozen-stored iCAR-ILC/N101.

TABLE 10

| Test items | Just before transport | | | Post transport | | |
|---|---|---|---|---|---|---|
| Tube Number | 1 | 28 | 71 | 7 | 37 | 70 |
| Viability (%) | 84 | 84 | 85 | 83 | 85 | 85 |
| Cell conc. | 1.84 | 2.00 | 1.89 | 1.87 | 1.85 | 1.86 |
| (×10⁻⁷/ml) | | | | | | |
| CAR | 99.6 | | | | | 99.1 |
| positivity (%) | | | | | | |
| Product purity (%) | 99.5 | | | | | 99.4 |
| IFN-γ production | | 514.7 | | 318.7 | | |
| (MFI) | | 445.0 | | | | |
| Endotoxin | Negative | | | Negative | | |
| Mycoplasma Bacteria | | | | | | |

| | | |
|---|---|---|
| Appearance | Nothing in particular | Nothing in particular |

The same production batch of iCAR-ILC/N101 is aliquoted (2×107/cell/tube) and frozen-stored. Three randomly picked cryotubes were transferred to a MEDi STAR cryoshipping box at the cell processing facility, and shipped to a shipper's facility 50 miles away. At the facility, the cryotubes are transferred to the gas phase of a liquid nitrogen tank. The cryotubes are transfer back to the cryoshipping box, air-transported to another shipper's facility 300 miles away, and shipped back to the cell processing facility as another air travel. The total distance was over 600 miles. The cells were tested for viability, cell concentration, CAR positivity, product purity, IFN-γ production, Endotoxin/Mycoplasma/bacterial pathogen detection, and appearance. All test results passed the quality standard as shown in the table 10.

Table 11 shows post-thaw stability of frozen-stored iCAR-ILC/N101.

TABLE 11

| Test items | Immediately after thaw | | | Left at room temp. | | |
|---|---|---|---|---|---|---|
| Tube Number | 1 | 28 | 71 | 22 | 23 | 24 |
| Post-thaw time | 0 min | | | 15 min | 30 min | 60 min |
| Viability (%) | 84 | 84 | 85 | 67 | 63 | 65 |
| Cell conc. | 1.84 | 2.00 | 1.89 | 1.44 | 1.28 | 1.42 |
| (×10⁻⁷/ml) | | | | | | |
| CAR | 99.6 | | | | | |
| positivity (%) | | | | | | |
| Product purity (%) | 99.5 | | | | | |
| IFN-γ production | | 514.7 | | 150.6 | 32.8 | 22.1 |
| (MFI) | | 445.0 | | | | |
| Endotoxin | Negative | | | | | |
| Mycoplasma | | | | | | |
| Bacteria | | | | | | |

| | |
|---|---|
| Appearance | Nothing in particular |

The same production batch of iCAR-ILC/N101 was aliquoted (2×10⁷/cell/tube) and frozen-stored. Three randomly picked cryotubes were thawed, and kept at room temperature. The cells were tested one tube at a time at 15, 30, and 90 minutes for viability, cell concentration, CAR positivity, product purity, IFN-γ production, Endotoxin/Mycoplasma/bacterial pathogen detection, and appearance. All test results passed the quality standard at 15 minutes, however, at 30 minutes, capability of IFN-γ production significantly reduced, although other test results passed the standard.

Table 12 shows stability of saline-diluted iCAR-ILC/N101 after thawing.

TABLE 12

| | | 0 min. | 60 min. | 90 min. |
|---|---|---|---|---|
| Fold dilution | Test Items | post dilution | post dilution | post dilution |
| 2 | Live cell | 2.12 × 10⁷ | 1.73 × 10⁷ | 2.00 × 10⁷ |
| | conc. | cells/mL | cells/mL | cells/mL |
| | Viability | 85% | 71% | 70% |
| 50 | Live cell | 1.77 × 10⁷ | 1.66 × 10⁷ | 1.82 × 10⁷ |
| | conc. | cells/mL | cells/mL | cells/mL |
| | Viability | 82% | 79% | 77% |

Frozen-stored iCAR-ILC/N101 cells were thawed, diluted in saline, and kept at room temperature. The cells were tested for live cell concentration and viability. Even at 90 minutes, live cell concentration and viability were not significantly reduced.

Table 13 shows standard tests for the final product (iCAR-ILC/N101).

TABLE

| | Test Items | Method |
|---|---|---|
| Standard test | Sterilization test | BacT/ALERT and Japar |
| | | Pharmacopoeia |
| | Mycoplasma test | PCR |
| | Bacterial endotoxin test | Kinetic colorimetry |
| | Purity of ILC | Flow cytometry |
| | Purity of tracer positive cells | Flow cytometry |
| | IFN-γ production ability | Flow cytometry |
| | Cell number | Trypan blue stain |
| | Cell viability | Trypan blue stain |
| | Expression of undifferentiated | Flow cytometry |
| | cell marker | |
| | Product appearance | Visual inspection |

As the standard tests, sterility, cell number, cell viability, cell phenotyping by flowcytometry, and IFN-γ secretion are examined, and the functional assays shown in table 13 are used as reference tests.

1. A method for producing a natural killer cell from an iPS (Induced pluripotent stem) cell, comprising steps of:
(i) contacting the iPS cell with a composition comprising a GSK-3 inhibitor and a ROCK inhibitor to obtain an embryoid body,
(ii) contacting the embryoid body with a composition comprising a TGFβ receptor inhibitor to obtain a hematopoietic progenitor cell,
(iii) culturing the hematopoietic progenitor cell to obtain a lymphocyte progenitor cell and
(iv) differentiating and expanding the lymphocyte progenitor cell to a natural killer cell.

2. The method according to claim 1, wherein the iPS cell expresses a tumor antigen specific chimeric antigen receptor (CAR).

3. The method according to claim 2, wherein the CAR expression is maintained or selected during differentiation process using a tracer gene and the CAR is stably expressed at the natural killer cell stage.

4. The method according to claim 2, wherein the tumor antigen is selected from a group consisting of GPC3, BCMA, PSMA, MUC1, HER2, Mesothelin, Lewis-Y, AXL, EGFR, Claudin18.2, B7-H3, NKG2D, GD2, EpCAM, ROBO-1, CD19, CD20, CD22, CD30, CD33, CD38, CD123, CD276, and CD269.

5. The method according to claim 2, wherein the iPS cell is undifferentiated from the iPS cell colonies with CAR.

6. The method according to claim 2, wherein the CAR is transduced into iPS cells using viral vectors, non-viral vectors, artificial chromosomes, or gene editing.

7. The method according to claim 6, wherein the viral vectors are Lentiviral vectors, retroviral vectors, adenoviral vectors or AAV vectors, and the non-viral vectors are piggyBac vectors.

8. The method according to claim 6, wherein the gene editing comprises using CRISPAR/CAS9, Talen, homologous recombination, or other gene editing tools.

9. The method according to claim 1, wherein the GSK-3 inhibitor is CHIR99021 and the ROCK inhibitor is Y-27632.

10. The method according to claim 1, wherein the TGFβ receptor inhibitor is SB431542.

11. The method according to claim 1, wherein the composition in step 2 further comprising VEGF, hbFGF and SCF.

12. The method according to claim 1, wherein the hematopoietic progenitor cell is cultured with a composition comprising 2-mercaptoethanol, insulin-transferrin-selenium, ascorbic acid-2-phosphate, SCF, TPO, IL-7, hFlt3L, SDF1α, and p38 inhibitor.

13. The method according to claim 1, wherein the p38 inhibitor is SB203580.

14. The method according to claim 1, wherein the lymphocyte progenitor cell is a CD7⁺CD45⁺ cell.

15. The method according to claim 1, wherein the lymphocyte progenitor cell is expanded on a feeder cell comprising a human PBMC.

16. The method according to claim 15, wherein the human PBMC is autologous or allogeneic.

17. A natural killer cell or a population thereof, produced by the method according to claim 1.

18. A natural killer cell population comprising cells that are CD7⁺CD45⁺ cells.

19. The natural killer cell population according to claim 18, wherein a percentage of CD7⁺CD45⁺ cells in the natural killer cell is more than 60% by cell number.

20. The natural killer cell population according to claim 18, wherein the cell is CD3⁻, CD4⁻, CD5⁻, CD8⁻, CD117⁺, CD337⁺, CD159a⁺, CD161⁺, CD336⁺, CD226⁺, and CD314⁺.

21. The natural killer cell population according to claim 18, wherein a contamination of undifferentiated iPSC is less than 0.01% by cell number in the natural killer cell.

22. A pharmaceutical composition comprising the natural killer cell or the population thereof according to claim 17.

23. The pharmaceutical composition according to claim 22, comprising a cryoprotective agent.

24. The pharmaceutical composition according to claim 22, comprising glucose, saline, dextran D, albuminar and dimethyl sulfoxide.

25. A method for treating cancer, comprising administrating the pharmaceutical composition according to claim 22.

26. The method according to claim 25, wherein the cancers are liver cancers, ovarian cancer, gastric cancers, lung cancers, prostate cancers, breast cancers, glioblastoma, colorectal cancers, esophageal cancers, head and neck cancers, cervical cancers, renal cancers, pediatric solid tumors, osteosarcoma, germ cell tumors, neuroblastoma, hematological malignancies, or multiple myeloma.
